(19) United States
(12) Patent Application Publication
(10) Pub. No.: US 2004/0005279 A1
(43) Pub. Date: Jan. 8, 2004

(54) Composition containing a semi-crystalline polymer and methods of use

(75) Inventors: Raluca Lorant, Thiais (FR); Paula Lennon, Lyon (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/347,687

(22) Filed: Jan. 22, 2003

Related U.S. Application Data
(60) Provisional application No. 60/356,172, filed on Feb. 14, 2002.

(30) Foreign Application Priority Data
Jan. 24, 2002 (FR) 0200882

(51) Int. Cl. A61K 7/44

(52) U.S. Cl. 424/60; 424/59; 424/70.9; 252/588; 252/589

(57) ABSTRACT

Use of a semi-crystalline polymer in a composition containing at least one organic UV screening agent. The polymer makes it possible to increase the sun protection factor (SPF). In addition, the semi-crystalline polymer makes it possible to introduce better dispersion of screening agents and confers, on the composition, good resistance to water (waterproof effect) and better spreading qualities. Composition for the photoprotection of keratinous substances, in particular for the protection of the skin and/or hair against solar radiation.

### REFERENCE TO PRIOR APPLICATIONS

This application claims priority to U.S. provisional application 60/356,172 filed Feb. 14, 2002, and French patent application 0200882 filed Jan. 24, 2002, both of which are incorporated herein by reference.

### SUMMARY OF THE INVENTION

The present application relates in general to the use of a semi-crystalline polymer in a composition (preferably intended for topical application and preferably intended for the protection of the skin and/or hair against UV radiation, in particular solar radiation) as an agent that increases the sun protection factor (SPF) of the composition. In addition, the semi-crystalline polymer of the invention makes it possible to better disperse screening agents and confers, on the composition, good resistance to water (waterproof effect) and better spreading qualities.

The present inveniton also relates to a composition (preferably for topical application) for the photoprotection of keratinous substances, preferably for the protection of the skin and/or hair against solar radiation, comprising, in a physiologically acceptable medium or support, at least one organic UV screening agent and a fatty phase comprising at least one semi-crystalline polymer, the organic UV screening agent not being a dibenzoylmethane derivative.

The present invention also relates to the use of a semi-crystalline for the purpose of improving the protection factor in ultraviolet radiation contributed by chemical screening agents.

These and other subjects and embodiments of the invention will become more clear upon an appreciation of both the background and detailed description of the invention.

### BACKGROUND OF THE INVENTION

It is known that light radiation with wavelengths of between 280 nm and 400 nm makes possible browning of the human epidermis and that, furthermore, radiation with wavelengths of between 280 nm and 320 nm, known under the name of UV-B radiation, causes skin bums and erythemas which can harm the development of natural tanning; this UV-B radiation must therefore be screened out.

It is also known that UV-A radiation, with wavelengths of between 320 nm and 400 nm, which causes browning of the skin, is capable of leading to a detrimental change in the skin, in particular in the case of sensitive skin or of skin continually exposed to solar radiation. UV-A radiation causes in particular a loss of elasticity of the skin and the appearance of wrinkles, resulting in premature ageing. It promotes the triggering of the erythemal reaction or accentuates this reaction in some subjects and can even be the cause of phototoxic or photoallergic reactions. It is therefore desirable to screen out UV-A radiation as well.

Numerous cosmetic compositions intended for photoprotection (UV-A and/or UV-B photoprotection) of the skin have been provided to date.

There exist on the market various types of sunscreen agents for screening out UV-A and UV-B radiation: pigments and chemical screening agents (or organic UV screening agents). These sunscreen agents must be able to absorb or block harmful solar radiation while remaining innocuous to the user.

For reasons of stability of these compositions and/or of toxicity on the skin or mucous membranes, it is not always possible to increase the amounts of chemical screening agents in order to increase the protection factor of antisun compositions. Consequently, the inventors have looked for other means for increasing the protection factor of these compositions.

Furthermore, the effectiveness of chemical or physical screening agents is often limited by problems of dispersion of these screening agents in the cosmetic compositions, the heterogeneity of the film formed at the surface of the skin and the low resistance to water.

In addition, many products, although pleasant when applied to the skin, exhibit the disadvantage of not being highly resistant to water, resulting in a loss in effectiveness of the protection over time, in particular in a wet environment, in the presence of water, of sweat or of sand, for example. To respond to this problem, antisun products are often formulated with a high level of oils or of waxes to introduce a waterproof effect. A disadvantage of this type of product is that of introducing a greasy feel that is unpleasant on use.

### DETAILED DESCRIPTION OF THE INVENTION

The inventors have discovered, surprisingly, that, by combining one or more screening agents with a semi-crystalline polymer, not only is good resistance to water obtained but also an improvement in the screening power of the screening agents and, for this reason, a marked improvement in the protection factor (SPF) of the composition comprising them, whatever the form (e.g., anhydrous gels, O/W and W/O emulsions, etc.)

The use of this polymer in this manner makes it possible to solve the problem of poor resistance to water in the case of emulsions. In addition, in any form, the addition of a small level of this polymer makes it possible to obtain higher protection factors without increasing the level of chemical screening agents. Furthermore, the presence of this polymer also makes possible better dispersion of the screening agents.

The compositions according to the invention comprising these polymers make it possible to obtain markedly improved textures and a reduced risk of discomfort in comparison with the conventional formulations, particularly those rich in screening agents (the term “rich” meaning at least 7% by weight of screening agents).

Accordingly, one embodiment of the invention is the use of a semi-crystalline polymer which is solid at ambient temperature and which has a melting point of less than 70° C., comprising a) a polymer backbone and b) at least one crystallizable organic side chain and/or at least one crystallizable organic block forming part of the backbone of the said polymer, the polymer having a number-average molecular mass (weight) of greater than or equal to 2 000, in a composition optionally comprising at least one liquid fatty phase and comprising at least one organic UV screening agent, this use preferably improving the photoprotective power of the composition.

Another embodiment of the invention is the use of a semi-crystalline polymer which is solid at ambient temperature and which has a melting point of less than 70° C., comprising a) a polymer backbone and b) at least one crystallizable organic side chain and/or at least one crystallizable organic block forming part of the backbone of the said polymer, the polymer having a number-average molecular mass (weight) of greater than or equal to 2 000, in the manufacture of a composition suitable for topical application and comprising at least one liquid fatty phase and at least one organic UV screening agent which is preferably intended for the protection of the skin, lips and/or hair against the harmful effects of UV radiation, this use preferably improving the protection introduced by the composition.

In the present application, the term “ambient temperature” is understood to mean a temperature of 25° C.

The photoprotective power is represented by the protection factor (SPF), which represents the screening power in the UV-A region. The determination of the UV-A protection factor is based on the method of evaluation of the immediate and persistent pigmentation induced by UV-A radiation (Persistent Pigment Darkening: PPD), described by Chardon et al. (Method for the UVA protection assessment of sunscreens based on residual immediate pigment darkening. 20th Annual Meeting of the American Society for Photobiology, Marco Island, Fla. (USA), Jun. 20-24, 1992), incorporated herein by reference.

Not only does the semi-crystalline polymer of the invention improve the protection factor but it also has the advantage of conferring better sensory qualities of spreading on the composition and makes it possible to obtain a matt and waterproof effect.

A preferred embodiment of the invention is thus a method of improving the photoprotective power and/or sensory qualities and/or matt effect and/or waterproof effect of a composition comprising at least one organic UV screening agent and optionally at least one liquid fatty phase, the method comprising combining in said composition at least one semi-crystalline polymer which is solid at 25° C. and which has a melting point of less than 70° C., comprising a) a polymer backbone and b) at least one crystallizable organic side chain and/or at least one crystallizable organic block forming part of the backbone of the polymer, the polymer having a number-average molecular weight of greater than or equal to 2 000. Here, the term “combining” includes any order of addition of components of the composition. Preferably, and with regard to improved photoprotective power, the polymer is present in an amount that increases the SPF of the composition above that measured in the absence of the polymer. In a highly preferred embodiment the UV screening agent is not a dibenzoylmethane derivative.

A further embodiment of the invention is a composition for topical application, preferably for the photoprotection of keratinous substances, such as the skin and/or hair, comprising, preferably in a physiologically acceptable medium, (1) at least one liquid fatty phase, (2) at least one organic UV screening agent, and (3) at least one semi-crystalline polymer which is solid at ambient temperature and which has a melting point of less than 70° C., comprising a) a polymer backbone and b) at least one crystallizable organic side chain and/or at least one crystallizable organic block forming part of the backbone of the polymer, the polymer having a number-average molecular mass (weight) of greater than or equal to 2 000, the UV screening agent not being a dibenzoylmethane derivative.

As the compositions of the invention may be intended for a topical application, they preferably comprise a physiologically acceptable medium, that is to say a medium compatible with all keratinous substances, such as the skin, nails, mucous membranes and hair or any other cutaneous region of the body.

The composition can constitute in particular a cosmetic composition. Generally, a cosmetic composition is intended to be brought into contact with the surface parts of the human body. An antisun cosmetic composition makes it possible to combat the effects of UV radiation on the surface layers of the skin and in particular the effects with regard to ageing of the skin (wrinkles and fine lines).

The semi-crystalline polymer used in the composition of the invention makes it possible to obtain higher protection factors without increasing the level of chemical screening agents and thus to improve the protection factor for a predetermined amount of screening agent. It is generally, but not necessarily, introduced into the liquid fatty phase (also referred to hereinafter as oily phase) when present.

The term “semi-crystalline polymer” is understood to mean, within the meaning of the invention, polymers comprising a crystallizable part, a pendant chain or a block in the backbone, and an amorphous part in the backbone and exhibiting a first-order reversible phase change temperature, in particular a melting point (solid-liquid transition). The term “polymers” is understood to mean, within the meaning of the invention, compounds comprising at least 2 repeat units, preferably at least 3 repeat units and more especially at least 10 repeat units. When the crystallizable part is a block of the polymer backbone, this crystallizable block has a different chemical nature from that of the amorphous block(s); in this case, the semi-crystalline polymer is a block polymer, for example of the diblock, triblock or multiblock type.

The semi-crystalline polymer or polymers of the invention advantageously have a number-average molecular mass (weight) \overline{M}n of greater than or equal to 2 000, for example ranging from 2 000 to 800 000, preferably from 3 000 to 500 000, for example from 4 000 to 150 000 and better still from 4 000 to 99 000, all values and subranges between these stated ranges being included as if specifically written out.

In the composition according to the invention, the semi-crystalline polymers are advantageously soluble in the fatty phase to at least 1% by weight at a temperature greater than their melting point. Apart from the crystallizable chains or blocks, the blocks of the polymers are amorphous. The term “crystallizable chain or block” is understood to mean, within the meaning of the invention, a chain or block which, if it were alone, would change reversibly from the amorphous state to the crystalline state, according to whether the temperature is above or below the melting point. A chain within the meaning of the invention is a group of atoms which is in the pendant or side position with respect to the backbone of the polymer. A block is a group of atoms belonging to the backbone, a group constituting one of the repeat units of the polymer.

The polymer backbone of the semi-crystalline polymers is preferably soluble in the liquid fatty phase.

Preferably, the semi-crystalline polymers used in the composition of the invention exhibit a melting point (or melting temperature) M.p. of less than 70° C., preferably of less than 50° C., this temperature being at least equal to the temperature of the keratinous substrate which has to receive the composition according to the invention. The semi-crystalline polymer has a melting point M.p. such that 25° C.≦M.p.≦70° C. and preferably 30° C.≦M.p.<50° C., all values and subranges between these ranges being included as if specifically written out. The melting point can be measured in particular by any known method and especially with a differential scanning calorimeter (D.S.C.).

Preferably, the crystallizable blocks or chains of the semi-crystalline polymers represent at least 30% of the total weight of each polymer and better still at least 40%. The semi-crystalline polymers of the invention with crystallizable blocks are block or multiblock polymers. They can be obtained by polymerization of monomers with reactive double bonds (i.e. ethylenic bonds) or by polycondensation. When the polymers of the invention are polymers with crystallizable side chains, these polymers are advantageously in the statistical or random form.

Preferably, the semi-crystalline polymers of the invention are synthetic in origin. In addition, they do not comprise a polysaccharide backbone.

Examples of semi-crystalline polymers which may be used in the invention include:

1. block copolymers of polyolefins with controlled crystallization, the monomers of which are disclosed in the document EP-A-951 897;

2. polycondensates and in particular the following polycondensates: aliphatic or aromatic polyesters and aliphatic/aromatic copolyesters;

3. homo- or copolymers carrying at least one crystallizable side chain and homo- or copolymers carrying, in the backbone, at least one crystallizable block, such as those disclosed in the document U.S. Pat. No. 5,156,911;

4. homo- or copolymers carrying at least one crystallizable side chain with fluorinated group(s), as disclosed in the document WO-A-01/19333;

5. and their blends.

In the last two cases (3 and 4), the crystallizable side chain or block or side chains or blocks are hydrophobic.

Preferred crystalline polymers with crystallizable side chains or carrying, in the backbone, at least one crystallizable block are described below.

A) Semi-crystalline Polymers With Crystallizable Side Chains

Mention may in particular be made of those defined in the documents U.S. Pat. No. 5,156,911 and WO-A-01/19333. These are homopolymers or copolymers comprising from 50 to 100% by weight of units resulting from the polymerization of one or more monomers carrying crystallizable hydrophobic side chain(s). These homo- or copolymers are of any nature, provided that they exhibit the conditions indicated below, with in particular the characteristic of being soluble or dispersible in the liquid fatty phase by heating above their melting point M.p. They can result:

from the polymerization, in particular free-radical polymerization, of one or more monomers with double bond(s) or ethylenic monomers reactive with respect to polymerization, namely with a vinyl, (meth)acrylic or allyl group;

from the polycondensation of one or more monomers carrying coreactive groups (carboxylic or sulphonic acid, alcohol, amine or isocyanate groups), such as, for example, polyesters, polyurethanes, polyethers, polyureas or polyamides.

a) Generally, the crystallizable units (chains or blocks) of the semi-crystalline polymers according to the invention originate from monomer(s) with crystallizable block(s) or chain(s) used for the manufacture of the semi-crystalline polymers. These polymers are chosen in particular from the homopolymers and copolymers resulting from the polymerization of at least one monomer with crystallizable chain(s) which can be represented by the formula X:

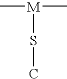

where M represents an atom of the polymer backbone, S represents a spacer and C represents a crystallizable group.

The crystallizable chains “—S—C” can be aliphatic or aromatic and optionally fluorinated or perfluorinated. “S” represents in particular a linear or branched or cyclic (CH₂)ₙ or (CH₂CH₂O)ₙ or (CH₂O) group, n being a integer ranging from 0 to 22. Preferably, “S” is a linear group. Preferably “S” and “C” are different.

When the crystallizable chains are aliphatic (alkyl) chains, they comprise at least 11 carbon atoms and at most 40 carbon atoms and better still at most 24 carbon atoms. They are in particular alkyl chains having at least 12 carbon atoms and they are preferably alkyl chains comprising from 14 to 24 carbon atoms (C₁₄-C₂₄). They can be hydrocarbonaceous alkyl chains (carbon and hydrogen atoms) or fluorinated or perfluorinated alkyl chains (carbon atoms, fluorine atoms and optionally hydrogen atoms). When they are fluorinated or perfluorinated alkyl chains, they comprise at least 11 carbon atoms, at least 6 carbon atoms of which are fluorinated.

Particular mention may be made, as examples of semi-crystalline polymers or copolymers with crystallizable chain(s), of those resulting from the polymerization of at least one monomer with a crystallizable chain chosen from saturated C₁₄-C₂₄ (C₁₄-C₂₄ means that the alkyl group comprises from 14 to 24 carbon atoms) alkyl (meth)acrylates; C₁₁-C₁₅ (alkyl group with 11 to 15 carbon atoms) perfluoroalkyl (meth)acrylates; C₁₄ to C₂₄ N-alkyl(meth)acrylamides, with or without a fluorine atom (alkyl group with 14 to 24 carbon atoms); vinyl esters with C₁₄ to C₂₄ (alkyl group with 14 to 24 carbon atoms) alkyl or perfluoroalkyl chains, with a perfluoroalkyl chain comprising at least 6 fluorine atoms; vinyl ethers with C₁₄ to C₂₄ (alkyl group with 14 to 24 carbon atoms) alkyl or perfluoroalkyl chains, with a perfluoroalkyl chain comprising at least 6 fluorine atoms; C₁₄ to C₂₄ (alkyl group with 14 to 24 carbon atoms) α-olefins, such as, for example, octadecene; C₁₄ to C₂₄ (alkyl group with 14 to 24 carbon atoms) para-alkylstyrenes; or their mixtures.

The term “alkyl” is understood to mean, within the meaning of the invention, a saturated group in particular comprising from 8 to 24 carbon atoms (C₈ to C₂₄), unless specifically mentioned.

When the polymers result from a polycondensation, the crystallizable hydrocarbonaceous and/or fluorinated chains as defined above are carried via a monomer which can be a diacid, a diol, a diamine or a diisocyanate.

When the polymers used in the composition of the invention are copolymers, they additionally comprise from 0 to 50% of Y or Z groups resulting from the copolymerization:

α) with Y, which is a polar or nonpolar monomer or a mixture of the two:

When Y is a polar monomer, it is either a monomer carrying polyoxyalkylenated (in particular oxyethylenated and/or oxypropylenated) groups, a hydroxyalkyl (meth)acrylate, such as hydroxyethyl acrylate, (meth)acrylamide, an N-alkyl(meth)acrylamide, an N,N-dialkyl(meth)acrylamide, such as, for example, N,N-diisopropylacrylamide, or N-vinylpyrrolidone (NVP) or N-vinylcaprolactam, or a monomeer carrying at least one carboxylic acid group, such as (meth)acrylic acid, crotonic acid, itaconic acid, maleic acid or fumaric acid, or carrying a carboxylic acid anhydride group, such as maleic anhydride, and their mixtures.

When Y is a nonpolar monomer, it can be an ester of the linear, branched or cyclic alkyl (meth)acrylate type, a vinyl ester, an alkyl vinyl ether, an α-olefin, styrene or styrene substituted by an alkyl group comprising from 1 to 10 carbon atoms (C₁ to C₁₀), such as α-methylstyrene, or a macromonomer of the polyorganosiloxane with vinyl unsaturation type.

β) with Z, which is a polar monomer or a mixture of polar monomers, Z having the same definition as the “polar Y” defined above.

Preferably, the semi-crystalline polymers with a crystallizable side chain are chosen from alkyl (meth)acrylate or alkyl(meth)acrylamide homopolymers with an alkyl group as defined above and in particular a C₁₄-C₂₄ alkyl group; copolymers of these monomers with a hydrophilic monomer, preferably different in nature from (meth)acrylic acid; and their blends. They can be, for example as copolymers, copolymers of alkyl (meth)acrylate or of alkyl(meth)acrylamide, with a C₁₄ to C₂₄ alkyl group, with N-vinylpyrrolidone or hydroxyethyl (meth)acrylate; or their blends.

B) Polymers Carrying, in the Backbone, at Least One Crystallizable Block

These are again polymers that are soluble or dispersible in the liquid fatty phase by heating above their melting point M.p. These polymers are in particular block copolymers composed of at least 2 blocks of different chemical natures, one of which is crystallizable.

Use may be made of, for example,:

1) The polymers defined in document U.S. Pat. No. 5,156,911;

2) Block copolymers of olefin or of cycloolefin with a crystallizable chain, such as those resulting from the block polymerization of:

cyclobutene, cyclohexene, cyclooctene, norbornene (that is to say, bicyclo[2.2.1]hept-2-ene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene or their mixtures,

with ethylene, propylene, 1-butene, 3-methyl-1-butene, 1-hexene, 4-methyl-1-pentene, 1-octene, 1-decene, 1-icosene or their mixtures.

These block copolymers can be in particular (ethylene/norbornene) block copolymers and (ethylene/propylene/ethylidenenorbornene) block terpolymers.

Use may also be made of those resulting from the block copolymerization of at least 2 C₂-C₁₆ α-olefins and better still C₂-C₁₂ α-olefins, such as those mentioned above, and in particular the block bipolymers of ethylene and of 1-octene.

3) Copolymers exhibiting at least one crystallizable block, the remainder of the copolymer being amorphous (at ambient temperature). These copolymers can, in addition, exhibit two crystallizable blocks of different chemical natures. The preferred copolymers are those which have, at ambient temperature, both a crystallizable block and a both hydrophobic and lipophilic amorphous block which are sequentially distributed; mention may be made, for example, of the polymers having one of the following crystallizable blocks and one of the following amorphous blocks:

Block crystallizable by nature: a) polyester, such as poly(alkylene terephthalate)s, b) polyolefin, such as polyethylenes or polypropylenes.

Amorphous and lipophilic block, such as amorphous polyolefins or copoly(olefin)s, for example poly(isobutylene), hydrogenated polybutadiene or hydrogenated poly(isoprene).

Mention may be made, as examples of such copolymers with a crystallizable block and with an amorphous block, of:

α) Poly(ε-caprolactone)-b-poly(butadiene) block copolymers, preferably used hydrogenated, such as those described in the article, “Melting behavior of poly(ε-caprolactone)-block-polybutadiene copolymers”, by S. Nojima, Macromolecules, 32, 3727-3734 (1999).

β) Block or multiblock hydrogenated poly(butylene terephthalate)-b-poly(isoprene) block copolymers, cited in the article, “Study of morphological and mechanical properties of PP/PBT”, by B. Boutevin et al., Polymer Bulletin, 34, 117-123 (1995).

γ) The poly(ethylene)-b-copoly(ethylene/propylene) block copolymers cited in the articles, “Morphology of semi-crystalline block copolymers of ethylene-(ethylene-alt-propylene)”, by P. Rangarajan et al., Macromolecules, 26, 4640-4645 (1993) and, “Polymer aggregates with crystalline cores: the system poly(ethylene)-poly(ethylene-propylene)”, P. Richter et al., Macromolecules, 30, 1053-1068 (1997).

δ) The poly(ethylene)-b-poly(ethylethylene) block copolymers cited in the general article, “Crystallization in block copolymers”, by I. W. Hamley, Advances in Polymer Science, vol. 148, 113-137 (1999).

The semi-crystalline polymers of the composition of the invention may be noncrosslinked or partially crosslinked provided that the degree of crosslinking is not harmful to their dissolution or dispersion in the liquid fatty phase by heating above their melting point. The crosslinking can then be chemical crosslinking, by reaction with a multifunctional monomer during the polymerization. It can also be physical crosslinking, which can then be due either to the establishment of bonds of hydrogen or dipolar type between groups carried by the polymer, such as, for example, dipolar interactions between carboxylate ionomers, these interactions being low in degree and carried by the backbone of the polymer, or to phase separation between the crystallizable blocks and the amorphous blocks carried by the polymer.

The semi-crystalline polymers of the composition according to the invention are preferably noncrosslinked.

Particular mention may be made, as specific example of semi-crystalline polymer that can be used in the composition according to the invention, of the Intelimer® products from Landec described in the brochure “Intelimer® polymers”. These polymers are in the solid form at ambient temperature (25° C.). They carry crystallizable side chains and exhibit monomer of the above formula X. Mention may in particular be made of “Landec IP22” with a melting point M.p. of 56° C., which is a product that is viscous at ambient temperature, impermeable and nonsticky.

Use may also be made of the semi-crystalline polymers disclosed in examples 3, 4, 5, 7 and 9 of the document U.S. Pat. No. 5,156,911, resulting from the copolymerization of acrylic acid and of C₅ to C₁₆ alkyl (meth)acrylate with an M.p. ranging from 20° C. to 35° C. and more particularly those resulting from the copolymerization:

of acrylic acid, of hexadecyl acrylate and of isodecyl acrylate in a 1/16/3 ratio,

of acrylic acid and of pentadecyl acrylate in a 1/19 ratio,

of acrylic acid, of hexadecyl acrylate and of ethyl acrylate in a 2.5/76.5/20 ratio,

of acrylic acid, of hexadecyl acrylate and of methyl acrylate in a 5/85/10 ratio,

of acrylic acid and of octadecyl methacrylate in a 2.5/97.5 ratio.

Use may also be made of the polymer “Structure O” sold by National Starch, such as that disclosed in the document U.S. Pat. No. 5,736,125 with an M.p. of 44° C., and of semi-crystalline polymers with crystallizable pendent chains comprising fluorinated groups, such as disclosed in Examples 1, 4, 6, 7 and 8 of the document WO-A-01/19333.

Use may also be made of the semi-crystalline polymers obtained by copolymerization of stearyl acrylate and of acrylic acid or of NVP as disclosed in the document U.S. Pat. No. 5,519,063 or EP-A-0 550 745 and more specifically those described in the polymer preparation Examples 1 and 2 below, with melting points of 40° C. and 38° C. respectively.

Use may also be made of the semi-crystalline polymers obtained by copolymerization of behenyl acrylate and of acrylic acid or of NVP as disclosed in the documents U.S. Pat. No. 5,519,063 and EP-A-0 550 745 and more especially those described in the polymer preparation Examples 3 and 4 below, with melting points of 60° C. and 58° C. respectively.

According to a specific embodiment of the invention, the semi-crystalline polymers used preferably do not comprise a carboxyl group.

The amount of semi-crystalline polymer in the composition of the invention can vary to a large extent according to the desired purpose and in particular a more or less extensive gelling of the oily phase. The amount of semi-crystalline polymers can range, for example, from 0.1 to 50% by weight, preferably from 0.5 to 20% by weight and better still from 1 to 10% by weight, with respect to the total weight of the composition, these ranges including all subranges and values therebetween as if specifically written out.

Organic UV Screening Agents (Or Sunscreen Agents)

The composition of the invention comprises at least one organic UV screening agent. This agent may be chosen from hydrophilic organic screening agents, lipophilic organic screening agents and their mixtures. According to a specific embodiment of the invention, one or more physical screening agents can be combined therein.

Useful examples of organic screening agents active in the UV-A and/or UV-B regions which can be used in the composition of the invention include the following, denoted below under their CTFA names:

para-Aminobenzoic Acid (PABA) Derivatives:

PABA,

Ethyl PABA,

Ethyl Dihydroxypropyl PABA,

Ethylhexyl Dimethyl PABA, sold in particular under the name “Escalol 507” by ISP,

Glyceryl PABA,

PEG-25 PABA, sold under the name “Uvinul P25” by BASF,

Salicylic Derivatives:

Homosalate, sold under the name “Eusolex HMS” by Rona/EM Industries,

Ethylhexyl Salicylate, sold under the name “Neo Heliopan OS” by Haarmann and Reimer,

Dipropyleneglycol Salicylate, sold under the name “Dipsal” by Scher,

TEA Salicylate, sold under the name “Neo Heliopan TS” by Haarmann and Reimer,

Dibenzoylmethane Derivatives:

Butyl Methoxydibenzoylmethane, sold in particular under the trade name “Parsol 1789” by Hoffmann-LaRoche,

Isopropyl Dibenzoylmethane,

Cinnamic Derivatives:

Ethylhexyl Methoxycinnamate (or Octyl Methoxycinnamate), sold in particular under the trade name “Parsol MCX” by Hoffmann-LaRoche,

Isopropyl Methoxycinnamate,

Isoamyl Methoxycinnamate, sold under the trade name “Neo Heliopan E 1000” by Haarmann and Reimer,

Cinoxate,

DEA Methoxycinnamate,

Diisopropyl Methyl Cinnamate,

Glyceryl Ethylhexanoate Dimethoxycinnamate,

β,β-Diphenylacrylate Derivatives:

Octocrylene (2-ethylhexyl α-cyano-β,β-diphenylacrylate), sold in particular under the trade name “Uvinul N539” by BASF,

Etocrylene, sold in particular under the trade name “Uvinul N35” by BASF,

Benzophenone Derivatives:

Benzophenone-1, sold under the trade name “Uvinul 400” by BASF,

Benzophenone-2, sold under the trade name “Uvinul D50” by BASF,

Benzophenone-3 or Oxybenzone, sold under the trade name “Uvinul M40” by BASF,

Benzophenone-4, sold under the trade name “Uvinul MS40” by BASF,

Benzophenone-5,

Benzophenone-6, sold under the trade name “Helisorb 11” by Norquay,

Benzophenone-8, sold under the trade name “Spectra-Sorb UV-24” by American Cyanamid,

Benzophenone-9, sold under the trade name “Uvinul DS-49” by BASF,

Benzophenone-12,

Benzylidenecamphor Derivatives:

3-Benzylidene camphor, manufactured under the name “Mexoryl SD” by Chimex,

4-Methylbenzylidene camphor, sold under the name “Eusolex 6300” by Merck,

Benzylidene Camphor Sulfonic Acid, manufactured under the name “Mexoryl SL” by Chimex,

Camphor Benzalkonium Methosulfate, manufactured under the name “Mexoryl SO” by Chimex,

Terephthalylidene Dicamphor Sulfonic Acid, manufactured under the name “Mexoryl SX” by Chimex,

Polyacrylamidomethyl Benzylidene Camphor, manufactured under the name “Mexoryl SW” by Chimex,

Phenylbenzimidazole Derivatives:

Phenylbenzimidazole Sulfonic Acid, sold in particular under the trade name “Eusolex 232” by Merck,

Benzimidazilate, sold under the trade name “Neo Heliopan AP” by Haarmann and Reimer,

Triazine Derivatives:

Anisotriazine, sold under the trade name “Tinosorb S” by Ciba-Geigy,

Ethylhexyl triazone, sold in particular under the trade name “Uvinul T150” by BASF,

Diethylhexyl Butamido Triazone, sold under the trade name “Uvasorb HEB” by Sigma 3V,

Phenylbenzotriazole Derivatives:

Drometrizole Trisiloxane, sold under the name “Silatrizole” by Rhodia Chimie,

Anthranilic Derivatives:

Menthyl anthranilate, sold under the trade name “Neo Heliopan MA” by Haarmann and Reimer,

Imidazoline Derivatives:

Ethylhexyl Dimethoxybenzylidene Dioxoimidazoline Propionate,

Benzalmalonate Derivatives:

Polyorganosiloxane with benzalmalonate functional groups, sold under the trade name “Parsol SLX” by Hoffmann-LaRoche,

and their mixtures.

The organic UV screening agents which are more particularly preferred are chosen from the following compounds:

Ethylhexyl Salicylate,

Butyl Methoxydibenzoylmethane,

Ethylhexyl Methoxycinnamate,

Octocrylene,

Phenylbenzimidazole Sulfonic Acid,

Terephthalylidene Dicamphor Sulfonic Acid,

Benzophenone-3,

Benzophenone-4,

Benzophenone-5,

4-Methylbenzylidene camphor,

Benzimidazilate,

Anisotriazine,

Ethylhexyl triazone,

Diethylhexyl Butamido Triazone,

Methylene Bis-benzotriazolyl Tetramethylbutylphenol,

Drometrizole Trisiloxane, and their mixtures.

The organic screening agent or agents can be present in any amount, preferably an amount ranging from 0.1 to 25% by weight, more preferably from 1 to 20% by weight and better still from 5 to 15% by weight, with respect to the total weight of the composition, these ranges including all subranges and values therebetween as if specifically written out.

Useful physical screening agents which can be added to the composition of the inventioninclude, for example, coated or uncoated metal oxide pigments and nanopigments, in particular titanium oxide, iron oxide, zirconium oxide, zinc oxide or cerium oxide, and their mixtures, it being possible for these oxides to be in the form of optionally coated micro- or nanoparticles (nanopigments).

According to the invention, the nanopigments may or may not be surface treated and can be chosen in particular from coated or uncoated titanium oxide (amorphous or crystalline in the rutile and/or anatase form), iron oxide, zirconium oxide, zinc oxide or cerium oxide nanopigments and their mixtures.

The treated nanopigments preferably are pigments which have been subjected to one or more surface treatments of chemical, electron, mechanochemical and/or mechanical nature with compounds such as described, for example, in Cosmetics & Toiletries, February 1990, Vol. 105, p. 53-64, such as amino acids, beeswax, fatty acids, fatty alcohols, anionic surfactants, lecithins, sodium, potassium, zinc, iron or aluminium salts of fatty acids, metal (titanium or aluminium) alkoxides, polyethylene, silicones, proteins (collagen, elastin), alkanolamines, silicon oxides, metal oxides, sodium hexametaphosphate, alumina or glycerol.

More particularly, the treated nanopigments can be titanium oxides treated with:

silica and alumina, such as the products “Microtitanium Dioxide MT 500 SA” and “Microtitanium Dioxide MT 100 SA” from Tayca, and the products “Tioveil Fin”, “Tioveil OP”, “Tioveil MOTG” and “Tioveil IPM” from Tioxide,

alumina and aluminium stearate, such as the product “Microtitanium Dioxide MT 100 T” from Tayca,

alumina and aluminium laurate, such as the product “Microtitanium Dioxide MT 100 S” from Tayca,

iron oxides and iron stearate, such as the product “Microtitanium Dioxide MT 100 F” from Tayca,

silica, alumina and silicone, such as the products “Microtitanium Dioxide MT 100 SAS”, “Microtitanium Dioxide MT 600 SAS” and “Microtitanium Dioxide MT 500 SAS” from Tayca,

sodium hexametaphosphate, such as the product “Microtitanium Dioxide MT 150 W” from Tayca,

octyltrimethoxysilane, such as the product “T-805” from Degussa,

alumina and stearic acid, such as the product “UVT-M160” from Kemira,

alumina and glycerol, such as the product “UVT-M212” from Kemira,

alumina and silicone, such as the product “UVT-M262” from Kemira.

The untreated titanium oxides can, for example, be those sold by Tayca under the trade names “Microtitanium Dioxide MT 500 B” or “Microtitanium Dioxide MT 600 B”.

The untreated zinc oxides can, for example, be those sold by Sumitomo under the name “Ultra Fine Zinc Oxide Powder”, by Presperse under the name “Finex 25”, by Ikeda under the name “MZO-25” or by Sunsmart under the name “Z-Cote”. The treated zinc oxides can, for example, be those sold by Sunsmart under the name “Z-Cote BP 1”.

The nanopigments can be introduced into the compositions according to the invention as such or in the form of a pigment paste, that is to say as a mixture with a dispersant, as disclosed, for example, in the document GB-A-2 206 339.

The physical screening agent or agents can be present in the compositions according to the invention in any amount, preferably in a proportion ranging from 0.1 to 20% by weight, preferably from 0.5 to 12% by weight and better still from 1 to 5% by weight, with respect to the total weight of the composition,m these ranges including all subranges and values therebetween as if specifically written out.

The fatty phase comprising the semi-crystalline polymer generally comprises at least one oil, namely an organic substance which is liquid at 20 to 25° C. and at atmospheric pressure (760 mmHg). Useful oils which can be used in the composition of the invention include, for example:

hydrocarbonaceous oils of animal origin, such as perhydrosqualene;

hydrocarbonaceous oils of vegetable origin, such as liquid triglycerides of fatty acids comprising from 4 to 10 carbon atoms, such as triglycerides of heptanoic or octanoic acids or, for example, sunflower, maize, soybean, gourd, grape seed, sesame, hazelnut, apricot, macadamia, arara, castor or avocado oils, triglycerides of caprylic/capric acids, such as those sold by Stéarineries Dubois or those sold under the names Miglyol 810, 812 and 818 by Dynamit Nobel, jojoba oil or karite butter oil;

synthetic esters and ethers, in particular of fatty acids, such as the oils of formulae R¹COOR² and R¹OR² in which R¹ represents the residue of a fatty acid comprising from 8 to 29 carbon atoms and R² represents a branched or unbranched hydrocarbonaceous chain comprising from 3 to 30 carbon atoms, such as, for example, Purcellin oil, isononyl isononanoate, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate; heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;

linear or branched hydrocarbons of mineral or synthetic origin, such as liquid paraffins, which may or may not be volatile, and their derivatives, liquid petrolatum, polydecenes or hydrogenated polyisobutene, such as Parleam® oil;

fatty alcohols having from 8 to 26 carbon atoms, such as cetyl alcohol, stearyl alcohol and their mixture (cetearyl alcohol), octyldodecanol, 2-butyloctanol, 2-hexyldecanol, 2-undecylpentadecanol, oleyl alcohol or linoleyl alcohol;

alkoxylated and in particular ethoxylated fatty alcohols, such as oleth-12;

partially hydrocarbonaceous and/or silicone-comprising fluorinated oils, such as these disclosed in the document JP-A-2-295912. Mention may also be made, as fluorinated oils, of perfluoromethylcyclopentane and perfluoro(1,3-dimethylcyclohexane), sold under the names of Flutec PC1® and Flutec PC1® by BNFL Fluorochemicals; perfluoro(1,2-dimethylcyclobutane); perfluoroalkanes, such as dodecafluoropentane and tetradecafluorohexane, sold under the names of PF 5050® and PF 5060® by 3M, or bromoperfluorooctane, sold under the name Foralkyl® by Atochem; nonafluoromethoxybutane, sold under the name MSX 4518® by 3M, and nonafluoroethoxyisobutane; or perfluoromorpholine derivatives, such as 4-(trifluoromethyl)perfluoromorpholine, sold under the name PF 5052® by 3M;

silicone oils, such as volatile or nonvolatile polymethylsiloxanes (PDMS) comprising a linear or cyclic silicone chain which are liquid or pasty at ambient temperature, in particular cyclopolydimethylsiloxanes (cyclomethicones), such as cyclohexasiloxane; polydimethylsiloxanes comprising pendant alkyl, alkoxy or phenyl groups or alkyl, alkoxy or phenyl groups at the silicone chain end, which groups have from 2 to 24 carbon atoms; or phenylated silicones, such as phenyl trimethicones, phenyl dimethicones, phenyltrimethylsiloxydiphenylsiloxanes, diphenyl dimethicones, diphenylmethyldiphenyltrisiloxanes, (2-phenylethyl)trimethylsiloxysilicates and polymethylphenylsiloxanes;

their mixtures.

The term “hydrocarbonaceous oil” in the list of the oils mentioned above is understood to mean any oil comprising predominantly carbon and hydrogen atoms and optionally ester, ether, fluorinated, carboxylic acid and/or alcohol groups.

Other fatty substances which can be present in the oily phase include, for example, fatty acids comprising from 8 to 30 carbon atoms, such as stearic acid, lauric acid, palmitic acid and oleic acid; waxes, such as lanolin wax, beeswax, carnauba or candelilla wax, paraffin or lignite waxes or microcrystalline waxes, ceresin or ozokerite, hydrogenated vegetable oils, such as hydrogenated jojoba oil, or synthetic waxes, such as polyethylene waxes or Fischer-Tropsch waxes; gums, such as silicone gums (dimethiconol); or silicone resins, such as trifluoromethyl C₁₋₄ alkyl dimethicone and trifluoropropyl dimethicone; and silicone elastomers, such as the products sold under the names “KSG” by Shin-Etsu, under the names “Trefil”, “BY29” or “EPSX” by Dow Corning or under the names “Gransil” by Grant Industries, and silicone elastomers comprising one or more oxyalkylenated and in particular oxyethylenated chains, such as the product sold under the name “KSG 21” by Shin-Etsu; and their mixtures.

These fatty substances can be chosen in a way varied by a person skilled in the art in order to prepare a composition having the desired properties, for example of consistency or of texture, in view of this disclosure.

The compositions used according to the invention can be provided in any form, such as those conventionally used for topical application and in particular in the form of oily solutions, of oil-in-water (O/W) or water-in-oil (W/O) or multiple emulsions, of oily gels, of liquid, pasty or solid anhydrous products, or of dispersions of a fatty phase in an aqueous phase using spherules, it being possible for these spherules to be polymeric nanoparticles, such as nanospheres and nanocapsules, or lipid vesicles of ionic and/or nonionic type. These compositions may be prepared according to the usual methods.

In addition, the compositions according to the invention can be more or less fluid and can have the appearance of a white or coloured cream, of an ointment, of a milk, of a lotion, of a serum, of a paste or of a foam. They can also be coloured when they are used as make-up products, generally by addition of coloured pigments or of dyes. They can optionally be applied to the skin in the form of an aerosol. They can also be provided in the solid form, for example in the form of a stick.

When the composition according to the invention is in the form of a water-in-oil (W/O) or oil-in-water (O/W) emulsion, the proportion of the fatty phase of the emulsion can preferably range from 5 to 80% by weight and more preferably from 5 to 50% by weight with respect to the total weight of the composition. The oils, the emulsifiers and the coemulsifiers used in the composition in the form of an emulsion are chosen from those used in the cosmetics or dermatological field. The emulsifier and the coemulsifier are generally preferably present in the composition in a proportion ranging from 0.3 to 30% by weight and preferably from 0.5 to 20% by weight with respect to the total weight of the composition. The emulsion can additionally comprise lipid vesicles.

The emulsions generally preferably comprise at least one emulsifier chosen from amphoteric, anionic, cationic or nonionic emulsifiers, used alone or as a mixture. The emulsifiers are appropriately chosen according to the emulsion to be obtained (W/O or O/W).

Useful emulsifying surfactants which can be used for the preparation of the W/O emulsions include, for example, sorbitan alkyl esters or ethers, glycerol alkyl esters or ethers or sugar alkyl esters or ethers; or silicone surfactants, such as dimethicone copolyols, for example the mixture of cyclomethicone and of dimethicone copolyol sold under the name “DC 5225 C” by Dow Corning, and alkyl dimethicone copolyols, for example Laurylmethicone copolyol, sold under the name “Dow Corning 5200 Formulation Aid” by Dow Corning, cetyl dimethicone copolyol, for example the product sold under the name Abil EM 90® by Goldschmidt, and the mixture of cetyl dimethicone copolyol, of polyglyceryl (4 mol) isostearate and of hexyl laurate sold under the name Abil WE 09 by Goldschmidt. One or more coemulsifiers which can advantageously be chosen from the group consisting of polyol alkyl esters can also be added thereto. Mention may in particular be made, as polyol alkyl esters, of glycerol and/or sorbitan esters and, for example, polyglyceryl isostearate, such as the product sold under the name Isolan GI 34 by Goldschmidt, sorbitan isostearate, such as the product sold under the name Arlacel 987 by ICI, the isostearate of sorbitan and of glycerol, such as the product sold under the name Arlacel 986 by ICI, and their mixtures.

Useful emulsifiers for O/W emulsions include, for example, nonionic emulsifers, such as oxyalkylenated (more particularly polyoxyethylenated) esters of fatty acids and of glycerol; oxyalkylenated esters of fatty acids and of sorbitan; oxyalkylenated (oxyethylenated and/or oxypropylenated) esters of fatty acids; oxyalkylenated (oxyethylenated and/or oxypropylenated) ethers of fatty alcohols; sugar esters, such as sucrose stearate; fatty alcohol and sugar ethers, in particular alkyl polyglucosides (APG), such as decyl glucoside and lauryl glucoside, sold, for example, by Henkel under the respective names Plantaren 2000 and Plantaren 1200, cetearyl glucoside, optionally as a mixture with cetearyl alcohol, sold, for example, under the name Montanov 68 by Seppic, under the name Tegocare CG90 by Goldschmidt and under the name Emulgade KE3302 by Henkel, and arachidyl glucoside, for example in the form of the mixture of arachidyl and behenyl alcohols and of arachidyl glucoside sold under the name Montanov 202 by Seppic. According to a specific preferred embodiment of the invention, the mixture of the alkyl polyglucoside as defined above with the corresponding fatty alcohol can be in the form of a self-emulsifying composition, as disclosed, for example, in the document WO-A-92/06778.

The compositions of the invention can also comprise any adjuvant used in the cosmetics or dermatological field in the usual concentrations. These adjuvants are preferably chosen in particular from hydrophilic or lipophilic gelling agents, preservatives, opacifying agents, emulsifiers, coemulsifiers, neutralizing agents, fragrances and their solubilizing or peptizing agents, colouring materials, fillers, and lipophilic or hydrophilic active principles. These adjuvants may be present in amounts preferably ranging from 0.01 to 30% of the weight of the composition.

Mention may be made, as hydrophilic gelling agents, of, for example, carboxyvinyl polymers, such as carbopols (carbomers) and Pemulens (acrylate/C₁₀-C₃₀ alkyl acrylate copolymer); polyacrylamides, such as, for example, the crosslinked copolymers sold under the names Sepigel 305 (C.T.F.A. name: polyacrylamide/C13-14 isoparaffin/Laureth 7) or Simulgel 600 (C.T.F.A. name: acrylamide/sodium acryloyldimethyltaurate copolymer/isohexadecane/polysorbate 80) by Seppic; optionally crosslinked and/or neutralized polymers and copolymers of 2-acrylamido-2-methylpropanesulphonic acid, such as the poly(2-acrylamido-2-methylpropanesulphonic acid) sold by Hoechst under the tradename “Hostacerin AMPS” (C.T.F.A. name: ammonium polyacryldimethyltauramide); cellulose derivatives, such as hydroxyethylcellulose; polysaccharides and in particular gums, such as xanthan gum; and their mixtures.

Mention may be made, as lipophilic gelling agents, of modified clays, such as hectorite and its derivatives, for example the products sold under the Bentone names.

Of course, these adjuvants should be of such a nature and concentration that they do not reduce the protection factor of the composition and do not destabilize the latter.

The compositions according to the invention make possible good protection and in particular good photoprotection (antisun protection) of the skin and/or hair and consequently have an effect on the photoinduced ageing of the skin and on the wrinkles and/or fine lines of the skin induced by UV radiation and in particular solar radiation and on the harmful effects for the health of the skin of UV radiation and in particular of solar radiation.

The invention consequently also relates to the use of a composition as defined above in the manufacture of a composition intended for the protection of the skin and/or hair against the harmful effects of UV radiation, in particular solar radiation.

The examples that follow serve to illustrate the invention without, however, exhibiting a limiting nature. In these examples, the compositions are given as % by weight, unless otherwise mentioned.

### I) EXAMPLES OF THE MANUFACTURE OF SEMI-CRYSTALLINE POLYMERS

### Example 1

### Acidic Polymer With a Melting Point of 40° C.

120 g of Parleam® oil (mineral oil) are introduced into a 1 l reactor equipped with a central anchor stirrer, a reflux condenser and a thermometer and are heated from ambient temperature to 80° C. over 45 min. The following mixture C₁ is introduced at 80° C. over 2 h:

40 g of cyclohexane+4 g of Triganox 141 [2,5-bis(2-ethylhexanoylperoxy)-2,5-dimethylhexane].

30 min after starting to run in the mixture C₁, the mixture C₂ is introduced over 1 h 30, which mixture C₂ is composed of:

190 g of stearyl acrylate+10 g of acrylic acid+400 g of cyclohexane.

After these two mixtures have finished being run in, reaction is allowed to take place for an additional 3 h at 80° C. and then all the cyclohexane present in the reaction medium is distilled off at atmospheric pressure.

The polymer, at 60% by weight of active material in Parleam® oil, is then obtained.

Its weight-average molecular mass M_{w} is 35 000, expressed as polystyrene equivalent, and its melting point M.p. is 40° C.±1° C., measured by D.S.C.

### Example 2

### Basic Polymer With a Melting Point of 38° C.

The same procedure as in Example 1 is applied, except that N-vinylpyrrolidone is used instead of acrylic acid.

The polymer obtained is at 60% by weight of active material in Parleam® oil, its weight-average molecular mass M_{w} is 38 000 and its M.p. is 38° C.

### Example 3

### Acidic Polymer With a Melting Point of 60° C.

The same procedure as in Example 1 is applied, except that behenyl acrylate is used instead of stearyl acrylate. The polymer obtained is at 60% by weight of active material in Parleam® oil. Its weight-average molecular mass M_{w} is 42 000 and its M.p. is 60° C.

### Example 4

### Basic Polymer With a Melting Point of 58° C.

The same procedure as in Example 2 is applied, except that behenyl acrylate is used instead of stearyl acrylate. The polymer obtained is at 60% by weight of active material in Parleam® oil. Its M_{w} is 45 000 and its M.p. is 58° C.

II) Composition Examples

### Example 5

### Fresh Gel With an SPF of 20

| | |
|---|---|
| Semi-crystalline polymer of Example 1 | 5% |
| Ethylhexyl methoxycinnamate (Parsol MCX) | 7% |
| Benzophenone-3 | 2% |
| Isohexadecane (oil) q.s. for | 100% |

The SPF of the gel obtained, measured in vitro, is 20.

### Example 6 (Comparative)

### Gel With an SPF of 15

| | |
|---|---|
| Ethylhexyl methoxycinnamate (Parsol MCX) | 7% |
| Benzophenone-3 | 2% |
| Isohexadecane (oil) q.s. for | 100% |

The SPF of the gel obtained, measured in vitro, is 15, although the amount of screening agent is identical to that in Example 1.

### Example 7

### W/O Emulsion With an SPF of 30 (Cream)

| Oily phase: | |
|---|---|
| Semi-crystalline polymer of Example 1 | 5% |
| Cetyl dimethicone copolyol (Abil EM90 from Goldschmidt) | 3% |
| Glyceryl isostearate (Isolan GI34 from Goldschmidt) | 1% |
| (coemulsifier) | |
| Mineral oil | 5% |
| Cyclomethicone | 3% |
| Ethylhexyl methoxycinnamate (Parsol MCX) | 7% |
| Titanium dioxide coated with stearic acid (Tiosperse Ultra | 10% |
| from Collaborative Laboratories) | |
| Aqueous phase: | |
| Glycerol | 5% |
| Preservative | q.s. |
| Water q.s. for | 100% |

Procedure: The two phases are homogenized under hot conditions (>80° C.) and then the emulsion is prepared with stirring by dispersion of the aqueous phase in the oily phase.

A stable emulsion is obtained, in the form of a cream which is pleasant to apply, with an SPF, measured in vitro, of 30.

### Example 8

### O/W Emulsion Gel With an SPF of 20

| Oily phase: | |
|---|---|
| Semi-crystalline polymer of Example 1 | 2% |
| Mineral oil | 6% |
| Cyclomethicone | 3% |
| Ethylhexyl methoxycinnamate (Parsol MCX) | 7% |
| Benzophenone-3 | 2% |
| Aqueous phase: | |
| Glycerol | 5% |
| Ammonium polyacryldimethyltauramide (Hostacerin AMPS | 1.5% |
| from Clariant) | |
| Preservative | q.s. |
| Water q.s. for | 100% |

Procedure: The AMPS gel is swollen in the aqueous phase under hot conditions. Furthermore, the components of the oily phase are homogenized at a temperature of approximately 80° C. and then the emulsion is prepared with stirring by dispersion of the oily phase in the aqueous phase.

An emulsion is obtained, which emulsion constitutes a cream which is fresh on application and which is pleasant to use. This cream has an SPF, measured in vitro, of 20.

### Example 9

### Day Cream (O/W Emulsion)

| Oily phase A: | |
|---|---|
| Semi-crystalline polymer of Example 1 | 2% |
| Mineral oil | 6% |
| Cyclomethicone | 3% |
| Ethylhexyl methoxycinnamate (Parsol MCX) | 7% |
| Aqueous phase B: | |
| Glycerol | 5% |
| Ammonium polyacryldimethyltauramide (Hostacerin AMPS | 1.5% |
| from Clariant) | |
| Mixture of arachidyl polyglucoside and of arachidyl and | 3% |
| behenyl alcohols (15/85) (Montanov 202 from Seppic) | |
| Glyceryl stearate | 1% |
| Preservative | q.s. |
| Triethanolamine | 0.4% |
| 3,3’-Terephthalylidene-10,10’-dicamphorsulphonic acid at | 2.1% |
| 33% in water (Mexoryl SX from Chimex) | |
| Water q.s. for | 100% |
| Phase C: | |
| 2-Phenylbenzimidazole-5-sulphonic acid (Eusolex 232 from | 2% |
| Rona/EM Industries) | |
| Triethanolamine | 1.7% |
| Water | 10% |

Procedure: The AMPS gel is swollen in aqueous phase B at 50° C. Furthermore, the components of the oily phase are homogenized at 60° C. and then the emulsion is prepared with stirring by dispersion of the oily phase in the aqueous phase. Furthermore, the components of phase C are homogenized until the screening agent has completely dissolved. This phase C is subsequently added to the (A+B) emulsion.

A beautiful white cream is obtained, which cream is soft and nongreasy to the touch and is capable of protecting from UV-A and UV-B radiation. This product can be used every day for protecting from the harmful effects of the sun. The in vitro SPF is 20.

All references, documents, applications, patents, publications, standards, tests, texts, etc., mentioned herein are specifically incorporated herein by reference.

The above description of the invention provides a full written description thereof, including the manner and process of making and using it, and enables one of ordinary skill in the art to make and use the invention as set forth above and in the following claims, all of which make up a part of the description. Further, one of ordinary skill is now able to both make and use a semi-crystalline polymer which is solid at ambient temperature and which has a melting point of less than 70° C., comprising a) a polymer backbone and b) at least one crystallizable organic side chain and/or one crystallizable organic block forming part of the backbone of the polymer, the polymer having a number-average molecular mass of greater than or equal to 2000, in a cosmetic composition for example intended for the protection of the skin, lips and/or hair against UV radiation and optionally comprising at least one liquid fatty phase and comprising at least one organic UV screening agent, for improving the photoprotective power of the composition, for example and for the protection of the skin, lips and/or hair against the harmful effects of UV radiation. Also fully described and enabled is a composition (preferably for topical application) wherein it comprises, preferably in a physiologically acceptable medium, (1) at least one liquid fatty phase, (2) at least one organic UV screening agent and (3) at least one semi-crystalline polymer which is solid at ambient temperature and which has a melting point of less than 70° C., comprising a) a polymer backbone and b) at least one crystallizable organic side chain and/or one crystallizable organic block forming part of the backbone of the polymer, the polymer having a number-average molecular mass of greater than or equal to 2000, the UV screening agent not being a dibenzoylmethane derivative. Also as fully described and enabled above, this composition, and more particularly the semi-crystalline polymer which is solid at ambient temperature and which has a melting point of less than 70° C., comprising a) a polymer backbone and b) at least one crystallizable organic side chain and/or one crystallizable organic block forming part of the backbone of the polymer, the polymer having a number-average molecular mass of greater than or equal to 2 000, can be used in the manufacture of a composition intended for the protection of the skin and/or hair against the harmful effects for the health of the skin of UV radiation, in particular solar radiation.

1. A method of improving the photoprotective power and/or sensory qualities and/or matt effect and/or waterproof effect of a composition comprising at least one organic UV screening agent and at least one liquid fatty phase, said method comprising combining in said composition at least one semi-crystalline polymer which is solid at 25° C. and which has a melting point of less than 70° C., comprising a) a polymer backbone and b) at least one crystallizable organic side chain and/or at least one crystallizable organic block forming part of the backbone of the polymer, the polymer having a number-average molecular weight of greater than or equal to 2 000.

2. The method according to claim 1, wherein said method improves the photoprotective power of said composition, and wherein the polymer is combined in an amount that increases the SPF of the composition as compared to the SPF of the composition in the absence of the polymer.

3. The method according to claim 1, wherein the polymer has a number-average molecular weight of 3 000 to 500 000.

4. The method according to claim 1, wherein the polymer is soluble in the fatty phase to at least 1% by weight at a temperature greater than its melting point.

5. The method according to claim 1, wherein the polymer has a melting point M.p of 25° C.≦M.p.<70° C.

6. The method according to claim 1, wherein the polymer is selected from the group consisting of:
block copolymers of polyolefins with controlled crystallization,
aliphatic or aromatic polyester and aliphatic/aromatic copolyester polycondensates,
homo- or copolymers carrying at least one crystallizable side chain and homo- or copolymers carrying, in the backbone, at least one crystallizable block,
homo- or copolymers carrying at least one crystallizable side chain with fluorinated group(s),
and their blends.

7. The method according to claim 1, wherein the polymer is selected from the group consisting of homopolymers and copolymers comprising from 50 to 100% by weight of units resulting from the polymerization of one or more monomers carrying crystallizable hydrophobic side chain(s).

8. The method according to claim 1, wherein the polymer is selected from the group consisting of the homopolymers and copolymers resulting from the polymerization of at least one monomer with crystallizable chain(s) of formula X: 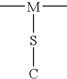
where M represents an atom of the polymer backbone, S represents a spacer and C represents a crystallizable group.

9. The method according to claim 1, wherein the polymer is selected from the group consisting of homopolymers and copolymers resulting from the polymerization of at least one monomer with a crystallizable chain chosen from saturated C₁₄-C₂₄ alkyl (meth)acrylates, C₁₁-C₁₅ perfluoroalkyl (meth)acrylates, C₁₄ to C₂₄ N-alkyl(meth)acrylamides, with or without a fluorine atom, vinyl esters with C₁₄ to C₂₄ alkyl or perfluoroalkyl chains, vinyl ethers with C₁₄ to C₂₄ alkyl or perfluoroalkyl chains, C₁₄ to C₂₄ α-olefins, para-alkylstyrenes with an alkyl group comprising from 12 to 24 carbon atoms, and their mixtures.

10. The method according to claim 1, wherein the polymer is selected from the group consisting of C₁₄ to C₂₄ alkyl (meth)acrylate or alkyl-(meth)acrylamide homopolymers; copolymers of these monomers with a hydrophilic monomer; and their blends.

11. The method according to claim 1, wherein the polymer is selected from the group consisting of copolymers of alkyl (meth)acrylate or of alkyl(meth)acrylamide, with a C₁₄ to C₂₄ alkyl group, with N-vinylpyrrolidone or hydroxyethyl (meth)acrylate; and their blends.

12. The method according to claim 1, wherein the polymer represents from 0.1 to 50% by weight with respect to the total weight of the composition.

13. The method according to claim 1, wherein the organic UV screening agent is selected from the group consisting of hydrophilic organic screening agents, lipophilic organic screening agents and their mixtures.

14. The method according to claim 1, wherein the organic UV screening agent is selected from the group consisting of para-aminobenzoic acids; salicylic compounds; dibenzoylmethanes; cinnamic compounds; β,β-diphenyl-acrylates; benzophenones; benzylidenecamphors; phenylbenzimidazoles; triazines; phenylbenzotriazoles; anthranilic compounds; imidazolines; benzalmalonates; and their mixtures.

15. The method according to claim 1, wherein the organic UV screening agent is selected from the group consisting of ethylhexyl salicylate, butyl methoxydibenzoylmethane, ethylhexyl methoxycinnamate, octocrylene, phenylbenzimidazole sulfonic acid, terephthalylidene dicamphor sulfonic acid, benzophenone-3, benzophenone-4, benzophenone-5, 4-methylbenzylidene camphor, benzimidazilate, anisotriazine, ethylhexyl triazone, diethylhexyl butamido triazone, methylene bis-benzotriazolyl tetramethylbutylphenol, drometrizole trisiloxane, and their mixtures.

16. The method according to claim 1, wherein the amount of organic screening agent(s) present in the composition is 0.1 to 25% by weight, with respect to the total weight of the composition.

17. The method according to claim 1, wherein the composition further comprises a physical screening agent selected from the group consisting of pigments and nanopigments formed of coated or uncoated metal oxides.

18. The method according to claim 17, wherein the pigments or nanopigments are selected from the group consisting of coated or uncoated titanium oxide, zinc oxide, iron oxide, zirconium oxide, cerium oxide and their mixtures.

19. The mentod according to claim 17, wherein the amount of physical screening agent(s) present in the composition is 0.1 to 20% by weight with respect to the total weight of the composition.

20. The method according to claim 1, wherein the fatty phase of the composition comprises at least one oil selected from the group consisting of hydrocarbonaceous oils of animal origin, hydrocarbonaceous oils of vegetable origin, synthetic esters and ethers, linear or branched hydrocarbons, fatty alcohols having from 8 to 26 carbon atoms, alkoxylated fatty alcohols, partially hydrocarbonaceous and/or silicone-comprising fluorinated oils, silicone oils, and their mixtures.

21. The method according to claim 1, wherein the composition is in the form of an oily solution, an emulsion, an oily gel, a liquid, a pasty or solid anhydrous product, or of a dispersion of a fatty phase in an aqueous phase having spherules.

22. A composition comprising: (1) at least one liquid fatty phase, (2) at least one organic UV screening agent, and (3) at least one semi-crystalline polymer which is solid at 25° C. and which has a melting point of less than 70° C., said polymer comprising a) a polymer backbone and b) at least one crystallizable organic side chain and/or at least one crystallizable organic block forming part of the backbone of the polymer, the polymer having a number-average molecular weight of greater than or equal to 2 000, wherein the UV screening agent is not a dibenzoylmethane.

23. A composition according to claim 22, wherein the polymer has a number-average molecular weight of 3 000 to 500 000.

24. A composition according to f claim 22, wherein the polymer is soluble in the fatty phase to at least 1% by weight at a temperature greater than its melting point.

25. A composition according to claim 22, wherein the polymer has a melting point M.p. such that 30° C.≦M.p.≦50° C.

26. A composition according to claim 22, wherein the polymer is selected from the group consisting of:
block copolymers of polyolefins with controlled crystallization,
aliphatic or aromatic polyester and aliphatic/aromatic copolyester polycondensates,
homo- or copolymers carrying at least one crystallizable side chain and homo- or copolymers carrying, in the backbone, at least one crystallizable block,
homo- or copolymers carrying at least one crystallizable side chain with fluorinated group(s),
and their blends.

27. A composition according to claim 22, wherein the polymer is selected from the group consisting of homopolymers and copolymers comprising from 50 to 100% by weight of units resulting from the polymerization of one or more monomers carrying crystallizable hydrophobic side chain(s).

28. A composition according to claim 22, wherein the polymer is selected from the group consisting of homopolymers and copolymers resulting from the polymerization of at least one monomer with crystallizable chain(s) of formula X: 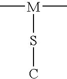
where M represents an atom of the polymer backbone, S represents a spacer and C represents a crystallizable group.

29. A composition according to claim 22, wherein the polymer is selected from the group consisting of homopolymers and copolymers resulting from the polymerization of at least one monomer with a crystallizable chain chosen from saturated C₁₄-C₂₄ alkyl (meth)acrylates, C₁₁-C₁₅ perfluoroalkyl (meth)acrylates, C₁₄ to C₂₄ N-alkyl(meth)acrylamides, with or without a fluorine atom, vinyl esters with C₁₄ to C₂₄ alkyl or perfluoroalkyl chains, vinyl ethers with C₁₄ to C₂₄ alkyl or perfluoroalkyl chains, C₁₄ to C₂₄ α-olefins, para-alkylstyrenes with an alkyl group comprising from 12 to 24 carbon atoms, and their mixtures.

30. A composition according to claim 22, wherein the polymer is selected from the group consisting of C₁₄ to C₂₄ alkyl (meth)acrylate or alkyl(meth)acrylamide homopolymers; copolymers of these monomers with a hydrophilic monomer; and their blends.

31. A composition according to claim 22, wherein the polymer is selected from the group consisting of copolymers of alkyl (meth)acrylate or of alkyl(meth)acrylamide, with a C₁₄ to C₂₄ alkyl group, with N-vinylpyrrolidone or hydroxyethyl (meth)acrylate; and their blends.

32. A composition according to claim 22, wherein the polymer is present in an amount of 0.1 to 50% by weight with respect to the total weight of the composition.

33. A composition according to claim 22, wherein the organic UV screening agent is selected from the group consisting of hydrophilic organic screening agents, lipophilic organic screening agents, and their mixtures.

34. A composition according to claim 22, wherein the organic UV screening agent is selected from the group consisting of para-aminobenzoic acids; salicylic compounds; cinnamic compounds; β,β-diphenylacrylates; benzophenones; benzylidenecamphors; phenylbenzimidazoles; triazines; phenylbenzotriazoles; anthranilic compounds; imidazolines; benzalmalonates; and their mixtures.

35. A composition according to claim 22, wherein the organic UV screening agent is selected from the group consisting of ethylhexyl salicylate, ethylhexyl methoxycinnamate, octocrylene, phenylbenzimidazole sulfonic acid, terephthalylidene dicamphor sulfonic acid, benzophenone-3, benzophenone-4, benzophenone-5, 4-methylbenzylidene camphor, benzimidazilate, anisotriazine, ethylhexyl triazone, diethylhexyl butamido triazone, methylene bis-benzotriazolyl tetramethylbutylphenol, drometrizole trisiloxane, and their mixtures.

36. A composition according to claim 22, wherein the amount of organic screening agent(s) present is 0.1 to 25% by weight with respect to the total weight of the composition.

37. A composition according to claim 22, further comprising a physical screening agent selected from the group consisting of pigments and nanopigments formed of coated or uncoated metal oxides.

38. A composition according to claim 37, wherein the pigments and nanopigments are selected from the group consisting of coated or uncoated titanium oxide, zinc oxide, iron oxide, zirconium oxide, cerium oxide and their mixtures.

39. A composition according to claim 37, wherein the physical screening agent(s) is present in 0.1 to 20% by weight with respect to the total weight of the composition.

40. A composition according to claims 22, wherein the fatty phase comprises at least one oil selected from the group consisting of hydrocarbonaceous oils of animal origin, hydrocarbonaceous oils of vegetable origin, synthetic esters and ethers, linear or branched hydrocarbons, fatty alcohols having from 8 to 26 carbon atoms, alkoxylated fatty alcohols, partially hydrocarbonaceous and/or silicone-comprising fluorinated oils, silicone oils, and their mixtures.

41. A composition according to claim 22, wherein it constitutes a cosmetic composition.

42. A method for the protection of the skin and/or hair comprising applying the composition of claim 22 to the skin and/or hair.
